(19) United States
(12) Patent Application Publication
(10) Pub. No.: US 2015/0025080 A1
(43) Pub. Date: Jan. 22, 2015

(54) SOLID DISPERSIONS OF SITAGLIPTIN AND PROCESSES FOR THEIR PREPARATION

(71) Applicants: Poonam Kaushik, Ghaziabad (IN); Ram Thaimattam, Hyderabad (IN); Mohan Prasad, Gurgaon (IN); Sudershan Kumar Arora, Gurgaon (IN)

(72) Inventors: Poonam Kaushik, Ghaziabad (IN); Ram Thaimattam, Hyderabad (IN); Mohan Prasad, Gurgaon (IN); Sudershan Kumar Arora, Gurgaon (IN)

(73) Assignee: RANBAXY LABORATORIES LIMITED, New Delhi, Delhi (IN)

(21) Appl. No.: 14/129,687

(22) PCT Filed: Jun. 29, 2012
(86) PCT No.: PCT/IB2012/053337

§ 371 Date: Sep. 12, 2014

(30) Foreign Application Priority Data
Jun. 29, 2011 (IN) 1842/DEL/2011

(51) Int. Cl. C07D 487/04

(52) U.S. Cl. CPC C07D 487/04
USPC 514/249; 544/350; 34/380; 34/284

(57) ABSTRACT

The present invention provides processes for the preparation of amorphous form of sitagliptin dihydrogen phosphate. It also provides a solid dispersion of sitagliptin dihydrogen phosphate, including in the amorphous form, and processes for its preparation.

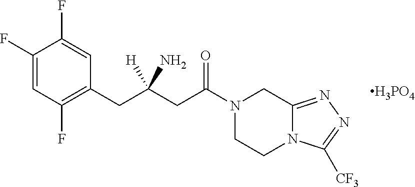

### FIELD OF THE INVENTION

The present invention provides processes for the preparation of amorphous form of sitagliptin dihydrogen phosphate. It also provides a solid dispersion of sitagliptin dihydrogen phosphate, including in the amorphous form, and a process for its preparation.

### BACKGROUND OF THE INVENTION

Sitagliptin dihydrogen phosphate monohydrate of Formula A, an orally-active inhibitor of the dipeptidyl peptidase-4 (DPP-4) enzyme, chemically designated as 7-[(3R)-3-amino-1-oxo-4-(2,4,5-trifluorophenyl)butyl]-5,6,7,8-tetrahydro-3-(trifluoromethyl)-1,2,4-triazolo[4,3-a]pyrazine phosphate (1:1) monohydrate is indicated as an adjunct to diet and exercise to improve glycemic control in adults with Type 2 diabetes mellitus.

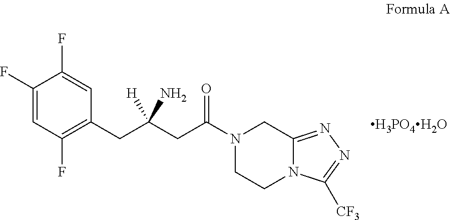 [SMILES: N[C@@H](CC(=O)N1CCn2c(nnc2C(F)(F)F)C1)Cc1cc(F)c(F)cc1F ; "·H3PO4·H2O"]

U.S. Pat. No. 6,699,871, in particular Example 7, provides a process for the preparation of sitagliptin base and its hydrochloride salt.

U.S. Pat. No. 7,326,708 provides a process for the preparation of sitagliptin dihydrogen phosphate monohydrate.

PCT Publication WO 2006/033848 provides a process for the preparation of amorphous sitagliptin dihydrogen phosphate which involves dissolving sitagliptin dihydrogen phosphate monohydrate in water and filtering to get a clear solution. The solution thus obtained was then frozen under a dry ice/methanol bath and then pulled under vacuum to remove the solvent to provide a fluffy, white amorphous solid of sitagliptin dihydrogen phosphate.

PCT Publication WO 2009/120746 provides a process for the preparation of sitagliptin dihydrogen phosphate in amorphous form. It involves slurrying sitagliptin base Form I in diethyl carbonate at 25° C. followed by the addition of phosphoric acid under stirring at 25° C. for 10 minutes. The reaction mixture is then filtered under vacuum to provide the amorphous form of sitagliptin phosphate. Another method involves slurrying sitagliptin base Form I in dimethyl carbonate at 50° C. followed by addition of phosphoric acid under stirring at 50° C. for 8 minutes. The reaction mixture is then filtered under vacuum to provide the amorphous form of sitagliptin phosphate.

Several processes are known in the literature for the preparation of sitagliptin or a salt thereof, for example, U.S. Publication No. 2009/0247532; PCT Publication Nos. WO 2010/097420; WO 2011/025932; WO 2010/122578; WO 2010/032264; WO 2010/131025; WO 2009/085990; WO 2005/020920; WO 2005/030127; WO 2004/085661; WO 2004/087650; WO 2006/065826; WO 2004/083212; and WO 2004/080958.

In the pharmaceutical industry there is a constant need to work on identifying different pharmaceutical compositions that positively affect the drug's dissolution profile, bioavailability, bioequivalence, stability, etc., which all play important roles in determining a drug's market acceptance and success.

In the case of sitagliptin too, there is a need for the development of pharmaceutical compositions with improved solubility, stability, excellent storage and handling stabilities, bioavailability, etc.

The present inventors have developed processes for the preparation of the amorphous form of sitagliptin dihydrogen phosphate. However, the present inventors found that sitagliptin dihydrogen phosphate in its amorphous form has a tendency to undergo crystallization at about 50% relative humidity (herein after “RH”) and 25° C. in a time period of about 4 days. Under certain circumstances, especially from a regulatory point of view, such interconversion is generally undesired.

The present inventors have surprisingly found that a solid dispersion of sitagliptin dihydrogen phosphate exhibits enhanced stability under humid conditions compared to amorphous sitagliptin dihydrogen phosphate, thus providing a viable solid dispersion product that eliminates the problem described above.

### SUMMARY OF THE INVENTION

A first aspect of the present invention provides a process for the preparation of amorphous sitagliptin dihydrogen phosphate which comprises:
a) obtaining a solution of sitagliptin dihydrogen phosphate;
b) removing the solvent from the solution obtained in step a) by spray drying; and
c) collecting sitagliptin dihydrogen phosphate in amorphous form.

A second aspect of the present invention provides a process for the preparation of amorphous sitagliptin dihydrogen phosphate which comprises:
a) obtaining a solution of sitagliptin dihydrogen phosphate;
b) removing the solvent from the solution obtained in step a) by agitated thin film drying; and
c) collecting sitagliptin dihydrogen phosphate in amorphous form.

A third aspect of the present invention provides an amorphous solid dispersion of sitagliptin dihydrogen phosphate.

A fourth aspect of the present invention provides a process for the preparation of a solid dispersion of sitagliptin dihydrogen phosphate which comprises:
a) combining sitagliptin dihydrogen phosphate with one or more pharmaceutically acceptable carriers; and
b) isolating a solid dispersion of amorphous sitagliptin dihydrogen phosphate.

A fifth aspect of the present invention provides a method of treating or preventing Type 2 diabetes mellitus which comprises administering to a patient in need thereof a therapeutically effective amount of solid dispersion of sitagliptin dihydrogen phosphate.

### DETAILED DESCRIPTION OF THE INVENTION

The sitagliptin dihydrogenphosphate prepared by any of the methods known in the art including those described in, for example, U.S. Pat. No. 7,326,708; U.S. Publication No. 2009/0247532; PCT Publication Nos. WO 2010/131025; WO 2004/083212; WO 2006/065826; WO 2010/097420; WO 2004/080958; WO 2004/087650; WO 2004/085661; WO 2005/072530; WO 2005/030127; WO 2005/020920; WO 2007/035198; WO 2008/000418; WO 2009/120746; WO 2006/033848; WO 2009/085990; WO 2010/032264; WO 2010/000469; WO 2010/012781; WO 2010/117738; WO 2010/092090; and WO 2010/122578; or amorphous sitagliptin dihydrogen phosphate prepared by the process of the present invention may be used as the starting material.

A first aspect of the present invention provides a process for the preparation of amorphous sitagliptin dihydrogen phosphate which comprises:
a) obtaining a solution of sitagliptin dihydrogen phosphate;
b) removing the solvent from the solution obtained in step a) by spray drying; and
c) collecting sitagliptin dihydrogen phosphate in amorphous form.

Embodiments of this aspect may include the following features:

A solution of sitagliptin dihydrogen phosphate can be obtained by treating sitagliptin dihydrogen phosphate with one or more solvent.

The term “solvent” includes any solvent or solvent mixture, including, for example, water, esters, alkanols, halogenated hydrocarbons, ketones, ethers, polar aprotic solvents, or mixtures thereof.

The esters may include one or more of ethyl acetate, n-propyl acetate, isopropyl acetate, and n-butyl acetate. Examples of alkanols include those primary, secondary and tertiary alcohols having from one to six carbon atoms. Suitable alkanol solvents include methanol, ethanol, n-propanol, isopropanol and butanol. Examples of halogenated hydrocarbons include dichloromethane, chloroform, and 1,2-dichloroethane. Examples of ketones include acetone, methyl ethyl ketone, and the like. Examples of ethers include diethyl ether, tetrahydrofuran, and the like. A suitable polar aprotic solvent includes one or more of N,N-dimethylformamide, N,N-dimethylacetamide, dimethylsulphoxide, acetonitrile and N-methylpyrrolidone.

Treating sitagliptin dihydrogen phosphate with one or more solvents may include adding, dissolving, slurrying, stirring, or a combination thereof.

Sitagliptin dihydrogen phosphate may be treated with solvent at a temperature of about 25° C. to reflux temperature.

The amount of solvent can be about 5 times to 20 times the quantity of sitagliptin dihydrogen phosphate.

The solution of sitagliptin dihydrogen phosphate obtained in step a) may be optionally clarified to remove foreign particulate matter or treated with activated charcoal to remove coloring and other related impurities. The solution of sitagliptin dihydrogen phosphate may be optionally concentrated to reduce the amount of solvent.

Step b) of removing the solvent from the solution obtained in step a) by spray drying involves feeding the solution obtained in step a) to a spray drying apparatus. The inlet and outlet temperatures, feed rate, and atomizer type can be adjusted to optimize output and particle size.

The air inlet temperature is preferably controlled at from about 70° C. to about 130° C. The outlet temperature is preferably controlled at from about 30° C. to about 65° C. An inert gas, for example nitrogen gas, can be used as a carrier gas.

After the drying process, the amorphous sitagliptin dihydrogen phosphate is collected from the spray dryer using techniques such as by scraping, or by shaking the container or other techniques specific to the equipment used.

A second aspect of the present invention provides a process for the preparation of amorphous sitagliptin dihydrogen phosphate which comprises:
a) obtaining a solution of sitagliptin dihydrogen phosphate;
b) removing the solvent from the solution obtained in step a) by agitated thin film drying; and
c) collecting sitagliptin dihydrogen phosphate in amorphous form.

Embodiments of this aspect may include the following features:

A solution of sitagliptin dihydrogen phosphate can be obtained by treating sitagliptin dihydrogen phosphate with one or more solvents.

Treating sitagliptin dihydrogen phosphate with one or more solvents may include adding, dissolving, slurrying, stirring, or a combination thereof.

The term “solvent” includes any solvent or solvent mixture, including, for example, water, esters, alkanols, halogenated hydrocarbons, ketones, ethers, polar aprotic solvents, or mixtures thereof.

The esters may include one or more of ethyl acetate, n-propyl acetate, isopropyl acetate, and n-butyl acetate. Examples of alkanols include those primary, secondary and tertiary alcohols having from one to six carbon atoms. Suitable alkanol solvents include methanol, ethanol, n-propanol, isopropanol and butanol. Examples of halogenated hydrocarbons include dichloromethane, chloroform, and 1,2-dichloroethane. Examples of ketones include acetone, methyl ethyl ketone, and the like. Examples of ethers include diethyl ether, tetrahydrofuran, and the like. A suitable polar aprotic solvent includes one or more of N,N-dimethylformamide, N,N-dimethylacetamide, dimethylsulphoxide, acetonitrile and N-methylpyrrolidone.

Sitagliptin dihydrogen phosphate may be treated with solvent at a temperature of about 25° C. to reflux temperature.

The amount of solvent can be about 5 times to 20 times the quantity of sitagliptin dihydrogen phosphate.

The solution of sitagliptin dihydrogen phosphate obtained in step a) may be optionally clarified to remove foreign particulate matter or treated with activated charcoal to remove coloring and other related impurities. The solution of sitagliptin dihydrogen phosphate may be optionally concentrated to reduce the amount of solvent.

Step b) of removing the solvent from the solution obtained in step a) by agitated thin film drying involves feeding the solution obtained in step a) to an agitated thin film dryer. The solvent is subsequently removed from the solution by agitated thin film drying by heating at a temperature of about 35° C. or above. The feeding rate of the solution is controlled in such a way as to facilitate the thin film formation and the evaporation rate. The rotor and vapor duct can have a sealing system so that the drying can preferably be carried out under vacuum. Vacuum operation also facilitates amorphous sitagliptin dihydrogen phosphate to be obtained without degradation.

The amorphous sitagliptin dihydrogen phosphate is collected from the agitated thin film dryer using techniques such as by scraping, or by shaking the container, or other techniques specific to the equipment used.

The amorphous sitagliptin dihydrogen phosphate can optionally be further dried under vacuum to obtain amorphous sitagliptin dihydrogen phosphate with desired residual solvent content.

A third aspect of the present invention provides a solid dispersion of sitagliptin dihydrogen phosphate.

The solid dispersion of sitagliptin dihydrogen phosphate of the present invention may be amorphous.

The solid dispersion of sitagliptin dihydrogen phosphate of the present invention comprises sitagliptin dihydrogen phosphate and one or more pharmaceutically acceptable carriers.

Pharmaceutically acceptable carrier is preferably a polymeric carrier, and more preferably is at least one from the group consisting of gelatines, ovalbumin, soybean proteins, gum arabic, non-sucrose fatty acid esters, starches, modified starches, cellulose, methylcellulose (MC), ethylcellulose (EC), hydroxyethylcellulose (HEC), hydroxypropylcellulose (HPC), hydroxypropylmethylcellulose (HPMC), polycarbophil, polyethylene glycol (PEG), polyethylene oxides, polyoxyalkylene derivatives, polymethacrylates, polyvinyl pyrrolidone (PVP), polyvinyl acetate (PVAc), PVP-vinylacetate-copolymer (PVP-VA), Kollidon® VA 64 (a vinylpyrrolidone-vinyl acetate copolymer), lactose, sorbitol, mannitol, maltitol, saccharose, isomalt, cyclodextrins such as cc-cyclodextrins, β-cyclodextrins, γ-cyclodextrins, hydroxyl-propyl-cyclodextrins, hydroxypropyl-β-cyclodextrin (HPβCD), sodium carboxymethyl cellulose, sodium alginate, xantham gum, locust bean gum (ceratonia), chitosan, cross-linked high amylase starch, cross-linked polyacrylic acid (carbopol), or a mixture thereof.

The amount of sitagliptin dihydrogen phosphate in the solid dispersion of the present invention ranges from about 0.1% to about 95% by weight relative to the total weight of the solid dispersion. In a preferred embodiment, the amount of sitagliptin dihydrogen phosphate ranges from about 1% to about 70%, more preferably from about 10% to about 50% by weight relative to the total weight of the solid dispersion.

The amorphous solid dispersion of sitagliptin dihydrogen phosphate of the present invention is stable during storage.

In a preferred embodiment, the polymeric carrier suitable for the preparation of a solid dispersion of sitagliptin dihydrogen phosphate is HPβCD.

The solid dispersion of sitagliptin dihydrogen phosphate with HPβCD is in the amorphous form.

The amorphous solid dispersion of sitagliptin dihydrogen phosphate with HPβCD of the present invention has a characteristic XRD pattern substantially as depicted in FIG. 4.

The amorphous solid dispersion of sitagliptin dihydrogen phosphate with HPβCD of the present invention has a characteristic XRD pattern substantially as depicted in FIG. 5.

The amorphous solid dispersion of sitagliptin dihydrogen phosphate with HPβCD of the present invention has a characteristic XRD pattern substantially as depicted in FIG. 6.

The amorphous solid dispersion of sitagliptin dihydrogen phosphate with HPβCD of the present invention is stable for at least 4 days when exposed to a temperature of about 25° C. and a relative humidity of about 50% and has a characteristic XRD pattern substantially as depicted in FIG. 13.

The amorphous solid dispersion of sitagliptin dihydrogen phosphate with HPβCD of the present invention is stable for at least 10 days when exposed to a temperature of about 25° C. and a relative humidity of 50% and has a characteristic XRD pattern substantially as depicted in FIG. 14.

The amorphous solid dispersion of sitagliptin dihydrogen phosphate with HPβCD of the present invention is stable for at least two months when kept in a double-sealed polybag at about 25° C. to 32° C. and has a characteristic XRD pattern substantially as depicted in FIG. 15.

In another preferred embodiment, the polymeric carrier suitable for the preparation of solid dispersion of sitagliptin dihydrogen phosphate is polyvinylpyrrolidone (PVP).

The solid dispersion of sitagliptin dihydrogen phosphate with polyvinylpyrrolidone is in amorphous form.

The amorphous solid dispersion of sitagliptin dihydrogen phosphate with polyvinylpyrrolidone (PVP) of the present invention has a characteristic XRD pattern substantially as depicted in FIG. 7.

The amorphous solid dispersion of sitagliptin dihydrogen phosphate with polyvinylpyrrolidone (PVP) of the present invention has a characteristic XRD pattern substantially as depicted in FIG. 8.

The amorphous solid dispersion of sitagliptin dihydrogen phosphate with polyvinylpyrrolidone (PVP) of the present invention has a characteristic XRD pattern substantially as depicted in FIG. 9.

The amorphous solid dispersion of sitagliptin dihydrogen phosphate with polyvinylpyrrolidone (PVP) of the present invention is stable for at least 4 days when exposed to a temperature of about 25° C. and a relative humidity of about 50% and has a characteristic XRD pattern substantially as depicted in FIG. 16.

The amorphous solid dispersion of sitagliptin dihydrogen phosphate with polyvinylpyrrolidone (PVP) of the present invention is stable for at least 10 days when exposed to a temperature of about 25° C. and a relative humidity of 50% and has a characteristic XRD pattern substantially as depicted in FIG. 17.

The amorphous solid dispersion of sitagliptin dihydrogen phosphate with polyvinylpyrrolidone (PVP) of the present invention is stable for at least two months when kept in double-sealed polybags at about 25° C. to 32° C. and has a characteristic XRD pattern substantially as depicted in FIG. 18.

A fourth aspect of the present invention provides a process for the preparation of a solid dispersion of sitagliptin dihydrogen phosphate which comprises:
a) combining sitagliptin dihydrogen phosphate with one or more pharmaceutically acceptable carriers; and
b) isolating solid dispersion of amorphous sitagliptin dihydrogen phosphate.

Combining sitagliptin dihydrogen phosphate with one or more pharmaceutically acceptable carriers may include adding, dissolving, slurrying, stirring or a combination thereof in a solvent at a temperature of about 25° C. to reflux temperature.

The term “solvent” includes any solvent or solvent mixture, including for example, water, esters, alkanols, halogenated hydrocarbons, ketones, ethers, polar aprotic solvents, or mixtures thereof.

The esters may include one or more of ethyl acetate, n-propyl acetate, isopropyl acetate, and n-butyl acetate. Examples of alkanols include those primary, secondary and tertiary alcohols having from one to six carbon atoms. Suitable alkanol solvents include methanol, ethanol, n-propanol, isopropanol and butanol. Examples of halogenated hydrocarbons include dichloromethane, chloroform, and 1,2-dichloroethane. Examples of ketones include acetone, methyl ethyl ketone, and the like. Examples of ethers include diethyl ether, tetrahydrofuran, and the like. A suitable polar aprotic solvent includes one or more of N,N-dimethylformamide, N,N-dimethylacetamide, dimethylsulphoxide, acetonitrile and N-methylpyrrolidone.

The pharmaceutically acceptable carrier is preferably a polymeric carrier, and more preferably is at least one from the group consisting of gelatines, ovalbumin, soybean proteins, gum arabic, non-sucrose fatty acid esters, starches, modified starches, cellulose, methylcellulose (MC), ethylcellulose (EC), hydroxyethylcellulose (HEC), hydroxypropylcellulose (HPC), hydroxypropylmethylcellulose (HPMC), polycarbophil, polyethylene glycol (PEG), polyethylene oxides, polyoxyalkylene derivatives, polymethacrylates, polyvinyl pyrrolidone (PVP), polyvinyl acetate (PVAc), PVP-vinylacetate-copolymer (PVP-VA), Kollidon® VA 64 (a vinylpyrrolidone-vinyl acetate copolymer), lactose, sorbitol, mannitol, maltitol, saccharose, isomalt, cyclodextrins such as cc-cyclodextrins, β-cyclodextrins, γ-cyclodextrins, hydroxyl-propyl-cyclodextrins, hydroxypropyl-β-cyclodextrin (HPβCD), sodium carboxymethyl cellulose, sodium alginate, xantham gum, locust bean gum (ceratonia), chitosan, cross-linked high amylase starch, cross-linked polyacrylic acid (carbopol), or a mixture thereof.

In a preferred embodiment, the polymeric carrier suitable for the preparation of solid dispersion of sitagliptin dihydrogen phosphate is polyvinylpyrrolidone (PVP) or HPβCD.

Step b) of isolating the solid dispersion of sitagliptin dihydrogen phosphate involves spray drying, lyophilization, agitated thin film drying or melt extrusion.

Isolating the solid dispersion of sitagliptin dihydrogen phosphate by spray drying involves feeding the solution obtained in step a) to a spray drying apparatus. The inlet and outlet temperatures, feed rate, and atomizer type can be adjusted to optimize output and particle size.

The air inlet temperature is preferably controlled at from about 70° C. to about 140° C. The outlet temperature is preferably controlled at from about 30° C. to about 65° C. An inert gas, for example nitrogen gas, can be used optionally as a carrier gas.

After the drying process, the solid dispersion of sitagliptin dihydrogen phosphate is collected from the spray dryer using techniques such as by scraping, or by shaking the container, or other techniques specific to the equipment used and optionally further dried under vacuum to obtain amorphous sitagliptin dihydrogen phosphate.

Isolating a solid dispersion of sitagliptin dihydrogen phosphate by agitated thin film drying involves feeding the solution obtained in step a) to an agitated thin film dryer. The solvent is subsequently removed from the solution by agitated thin film drying by heating at a temperature of about 35° C. or above. The feeding rate of the solution is controlled in such a way to facilitate the thin film formation and the evaporation rate. The rotor and vapor duct can have a sealing system so that the drying can preferably be carried out under vacuum. Vacuum operation also facilitates solid dispersion of sitagliptin dihydrogen phosphate to be obtained without degradation.

The solid dispersion of sitagliptin dihydrogen phosphate is collected from the agitated thin film dryer using techniques such as by scraping, or by shaking the container or other techniques specific to the equipment used.

The solid dispersion of sitagliptin dihydrogen phosphate may optionally be micronized to obtain the micronized amorphous solid dispersion of sitagliptin dihydrogen phosphate by suitable methods known in the art.

The solid dispersion of sitagliptin dihydrogen phosphate isolated by any of the methods above may be formulated into pharmaceutical compositions by further processing with one or more pharmaceutically inert excipients such as one or more of diluents, binders, disintegrants, coloring agents, flavoring agents, stabilizers, lubricants/glidants and plasticizers.

A fifth aspect of the present invention provides a method of treating or preventing Type 2 diabetes mellitus which comprises administering to a patient in need thereof a therapeutically effective amount of solid dispersion of sitagliptin dihydrogen phosphate.

### BRIEF DESCRIPTION OF THE FIGURES

FIG. 1 depicts the X-Ray Powder Diffractogram (XRPD) of amorphous sitagliptin dihydrogen phosphate, prepared as per Example 1.

FIG. 2 depicts the X-Ray Powder Diffractogram (XRPD) of amorphous sitagliptin dihydrogen phosphate, prepared as per Example 2.

FIG. 3 depicts the X-Ray Powder Diffractogram (XRPD) of amorphous sitagliptin dihydrogen phosphate, prepared as per Example 3.

FIG. 4 depicts the X-Ray Powder Diffractogram (XRPD) of an amorphous solid dispersion of sitagliptin dihydrogen phosphate with HPβCD, prepared as per Example 4.

FIG. 5 depicts the X-Ray Powder Diffractogram (XRPD) of an amorphous solid dispersion of sitagliptin dihydrogen phosphate with HPβCD, prepared as per Example 5.

FIG. 6 depicts the X-Ray Powder Diffractogram (XRPD) of an amorphous solid dispersion of sitagliptin dihydrogen phosphate with HPβCD, prepared as per Example 6.

FIG. 7 depicts the X-Ray Powder Diffractogram (XRPD) of an amorphous solid dispersion of sitagliptin dihydrogen phosphate with polyvinylpyrrolidone (PVP), prepared as per Example 7.

FIG. 8 depicts the X-Ray Powder Diffractogram (XRPD) of an amorphous solid dispersion of sitagliptin dihydrogen phosphate with polyvinylpyrrolidone (PVP), prepared as per Example 8.

FIG. 9 depicts the X-Ray Powder Diffractogram (XRPD) of an amorphous solid dispersion of sitagliptin dihydrogen phosphate with polyvinylpyrrolidone (PVP), prepared as per Example 9.

FIG. 10 depicts the X-Ray Powder Diffractogram (XRPD) of amorphous sitagliptin dihydrogen phosphate stored at 50% RH and 25° C. for 4 days.

FIG. 11 depicts the X-Ray Powder Diffractogram (XRPD) of amorphous sitagliptin dihydrogen phosphate stored at 50% RH and 25° C. for 10 days.

FIG. 12 depicts the X-Ray Powder Diffractogram (XRPD) of amorphous sitagliptin dihydrogen phosphate stored in a double-sealed polybag at 25° C. to 32° C. after two months.

FIG. 13 depicts the X-Ray Powder Diffractogram (XRPD) of an amorphous solid dispersion of sitagliptin dihydrogen phosphate with HPβCD stored at 50% RH and 25° C. for 4 days.

FIG. 14 depicts the X-Ray Powder Diffractogram (XRPD) of an amorphous solid dispersion of sitagliptin dihydrogen phosphate with HPβCD stored at 50% RH and 25° C. for 10 days.

FIG. 15 depicts the X-Ray Powder Diffractogram (XRPD) of an amorphous solid dispersion of sitagliptin dihydrogen phosphate with HPβCD stored in a double-sealed polybags at 25° C. to 32° C. for two months.

FIG. 16 depicts the X-Ray Powder Diffractogram (XRPD) of an amorphous solid dispersion of sitagliptin dihydrogen phosphate with polyvinyl pyrrolidone (PVP) stored at 50% RH and 25° C. for 4 days.

FIG. 17 depicts the X-Ray Powder Diffractogram (XRPD) of an amorphous solid dispersion of sitagliptin dihydrogen phosphate with polyvinyl pyrrolidone (PVP) stored at 50% RH and 25° C. for 10 days.

FIG. 18 depicts the X-Ray Powder Diffractogram (XRPD) of an amorphous solid dispersion of sitagliptin dihydrogen phosphate with polyvinyl pyrrolidone (PVP) stored in a double sealed polybag at 25° C. to 32° C. for two months.

The X-ray powder diffractograms (XRPD) of the samples were determined by using Instrument: PANalytical; Mode: Expert PRO; Detector: Xcelerator; ScanRange: 3-40; Step size: 0.02; Range: 3-40° 2 theta; CuKα radiation at 45 kV.

While the present invention has been described in terms of its specific embodiments, certain modifications and equivalents will be apparent to those skilled in the art and are intended to be included within the scope of the present invention.

### EXAMPLES

### Example 1

### Preparation of Amorphous Sitagliptin Dihydrogen Phosphate

Sitagliptin dihydrogen phosphate (5.02 g) was dissolved in methanol (250 ml) by heating at about 65° C. The solution was spray dried under the following conditions:

Air Inlet temperature: 100° C.

Air Outlet temperature: 49° C.

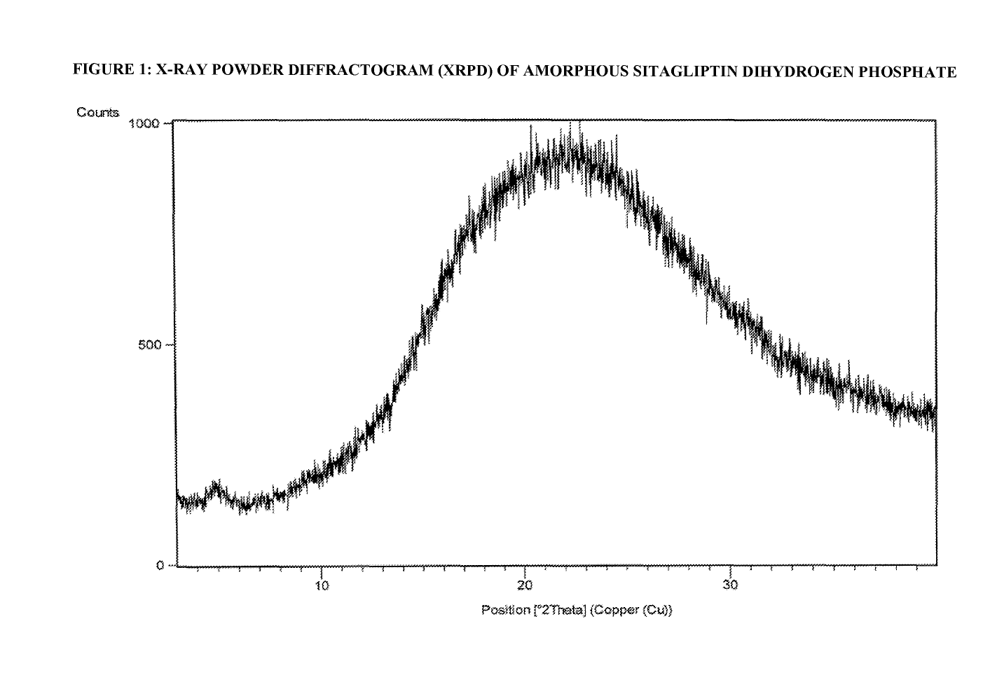

The solid so obtained was collected from the spray dryer and dried in a vacuum tray drier at 50° C. for 4 hours to obtain the titled compound having an XRPD pattern as depicted in FIG. 1.

### Yield: 2.89 g

### Example 2

### Preparation of Amorphous Sitagliptin Dihydrogen Phosphate

Sitagliptin dihydrogen phosphate (10.03 g) was dissolved in water (100 ml) by heating at about 65° C. The solution was spray dried under the following conditions:

Air Inlet temperature: 130° C.

Air Outlet temperature: 61° C.

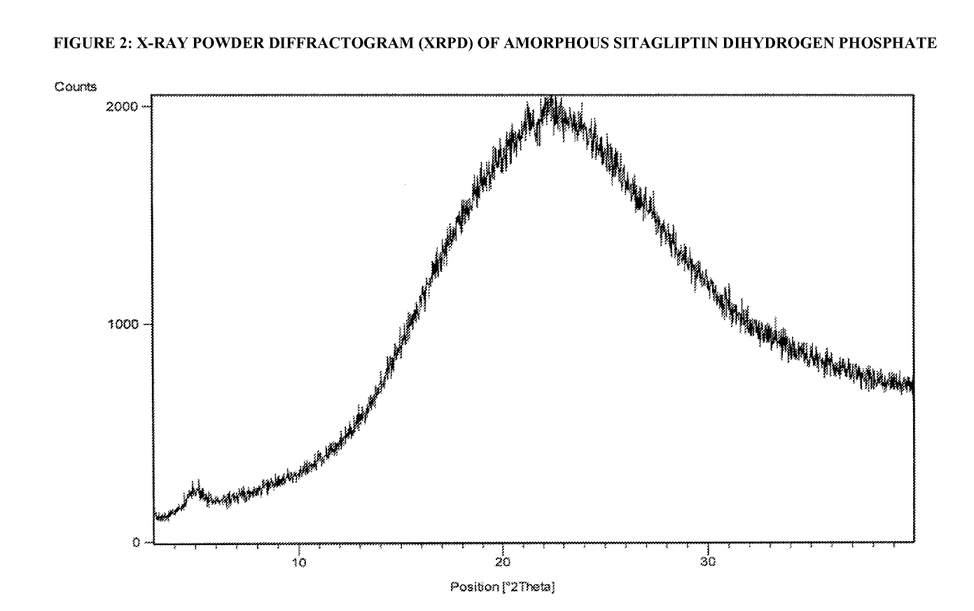

The solid so obtained was collected from the spray dryer and dried in a vacuum tray drier at 60° C. for 6 hours to obtain the titled compound having an XRPD pattern as depicted in FIG. 2.

### Yield: 6.29 g

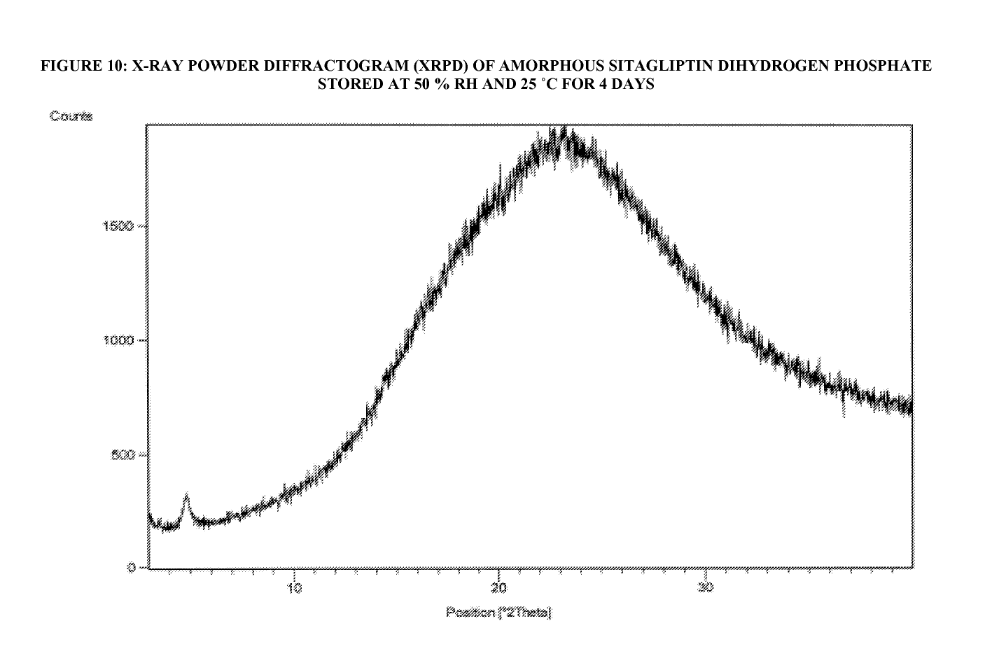

0.52 g of the product obtained as per Example 2 was stored in double sealed polybags in a humidity chamber maintained at 50% RH and 25° C. for 4 days to evaluate the stability. The XRPD pattern of the compound stored at 50% RH and 25° C. for 4 days is depicted in FIG. 10.

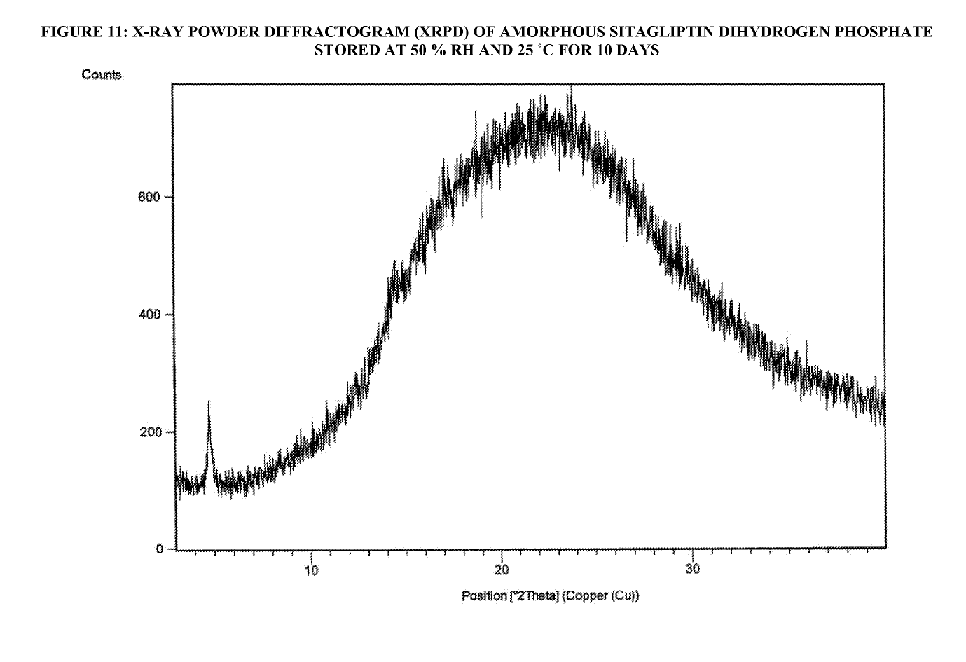

0.54 g of the product obtained as per Example 2 was stored in double sealed polybags in a humidity chamber maintained at 50% RH and 25° C. for 10 days to evaluate the stability. The XRPD pattern of the compound stored at 50% RH and 25° C. for 10 days is depicted in FIG. 11.

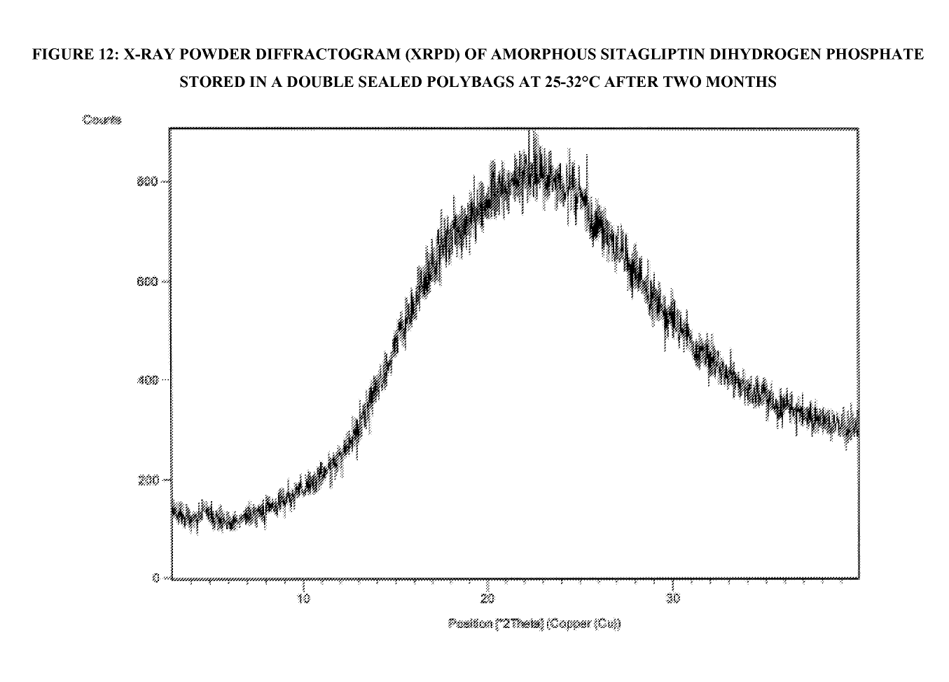

The remaining product obtained as per Example 2 was stored in double-sealed polybags at 25° C. to 32° C. for two months to evaluate the stability. The XRPD pattern of the compound stored in a double sealed polybag at 25° C. to 32° C. after two months is depicted in FIG. 12.

### Example 3

### Preparation of Amorphous Sitagliptin Dihydrogen Phosphate

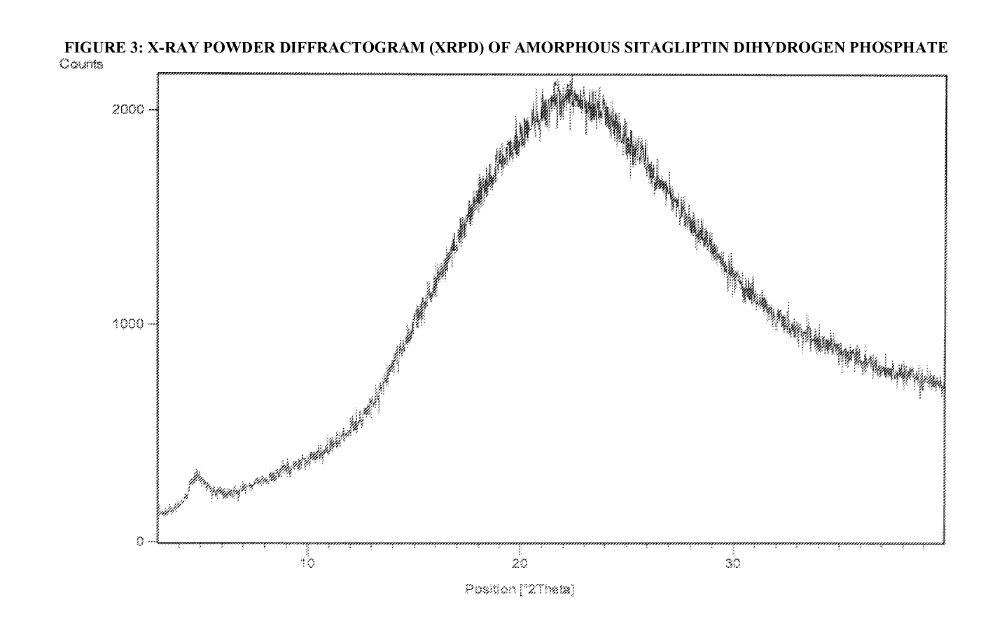

Sitagliptin dihydrogen phosphate (1.50 g) was dissolved in 20 ml water. The solvent was distilled off on a Buchi rotovap set at ˜75° C. and 250 rpm under vacuum. The solid so obtained was collected and dried in a vacuum tray drier at 50° C. for 4 hours to obtain the titled compound having an XRPD pattern as depicted in FIG. 3.

### Yield: 1.12 g

### Example 4

### Preparation of Amorphous Solid Dispersion of Sitagliptin Dihydrogen Phosphate with Hpβcd

Sitagliptin dihydrogen phosphate (5.02 g) and HPβCD (5.01 g) were dissolved in 100 ml water by heating at about 65° C. The solution thus obtained was spray dried under the following conditions:

Air Inlet temperature: 130° C.

Air Outlet temperature: 63° C.

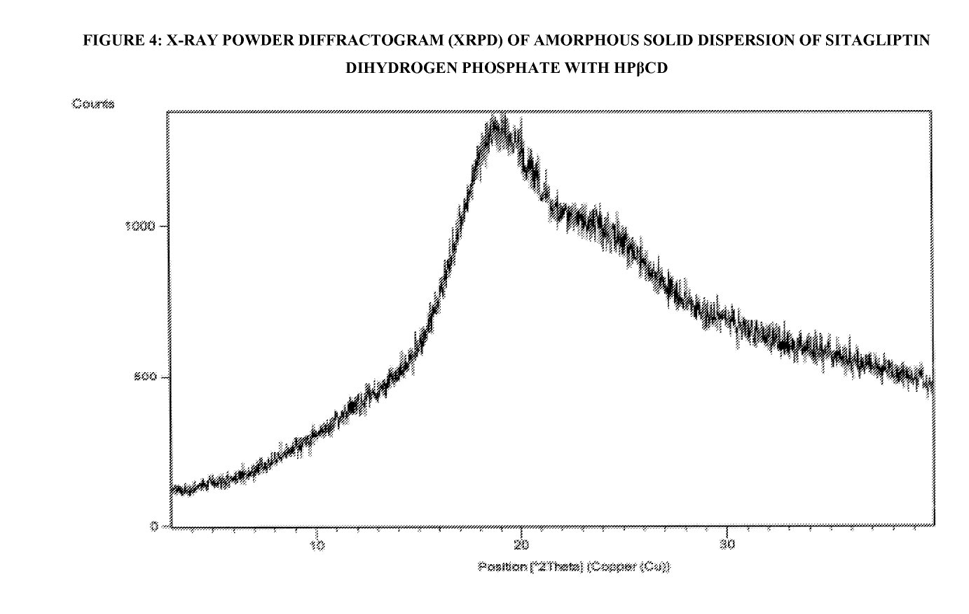

The solid so obtained was collected from the spray dryer and dried in a vacuum tray drier at 60° C. for 6 hours to obtain the titled compound having an XRPD pattern as depicted in FIG. 4.

### Yield: 6.26 g

### Example 5

### Preparation of Amorphous Solid Dispersion of Sitagliptin Dihydrogen Phosphate with HPβCD

Sitagliptin dihydrogen phosphate (5.04 g) and HPβCD (5.09 g) were dissolved in water (100 ml) by heating at about 65° C. The solution thus obtained was spray dried under the following conditions:

Air Inlet temperature: 130° C.

Air Outlet temperature: 61° C.

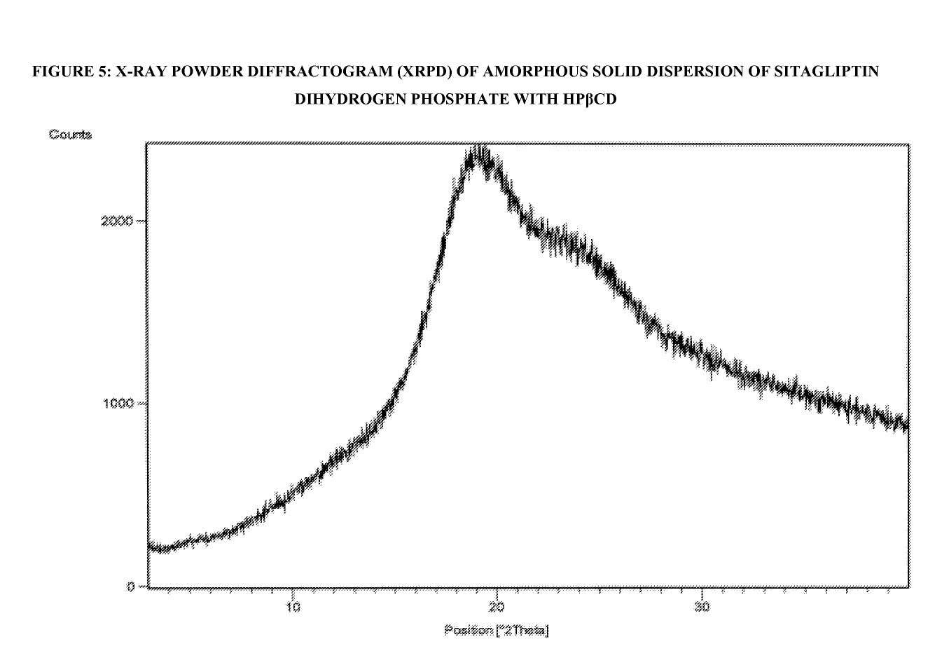

The solid so obtained was collected from the spray dryer and dried in a vacuum tray drier at 60° C. for 6 hours to obtain the titled compound having an XRPD pattern as depicted in FIG. 5.

### Yield: 5.49 g

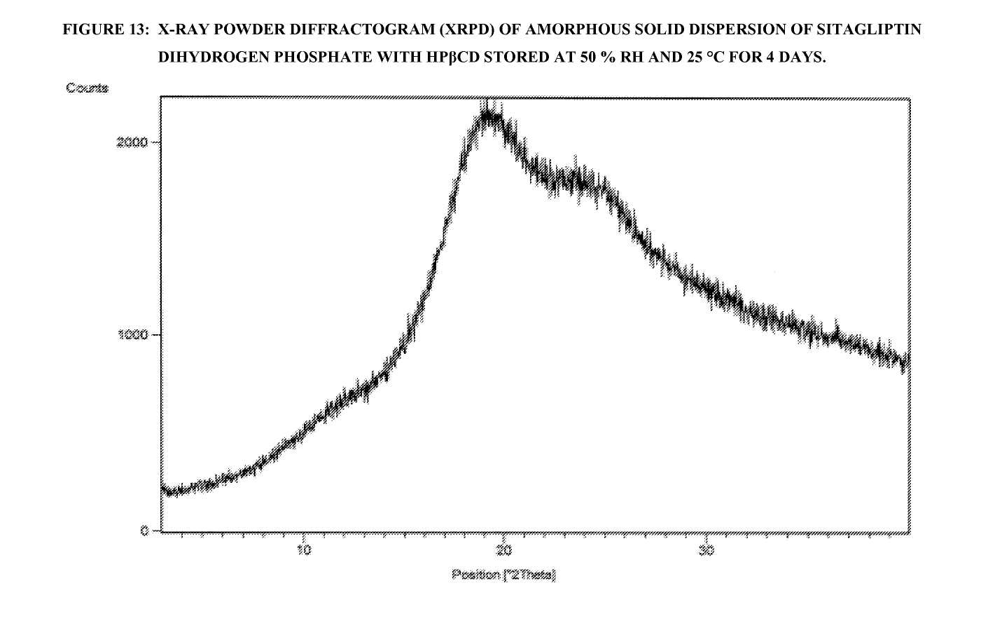

0.54 g of the product obtained as per Example 5 was stored in double-sealed polybags in a humidity chamber maintained at 50% RH and 25° C. for 4 days to evaluate the stability. The XRPD pattern of the product stored at 50% RH and 25° C. for 4 days is depicted in FIG. 13.

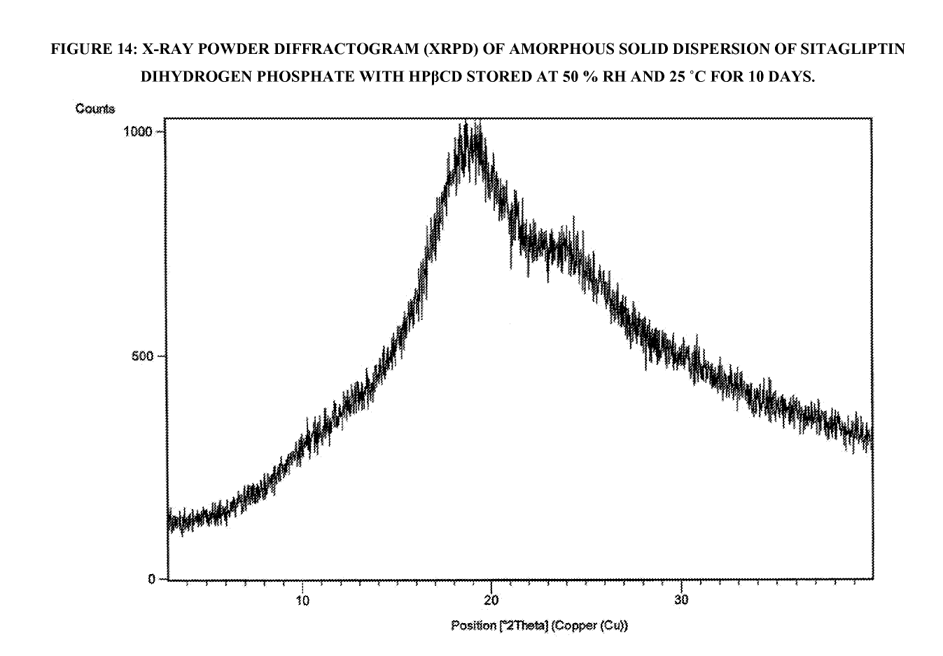

0.53 g of the product obtained as per Example 5 was stored in double-sealed polybags in a humidity chamber maintained at 50% RH and 25° C. for 10 days to evaluate the stability. The XRPD pattern of the product stored at 50% RH and 25° C. for 10 days is depicted in FIG. 14.

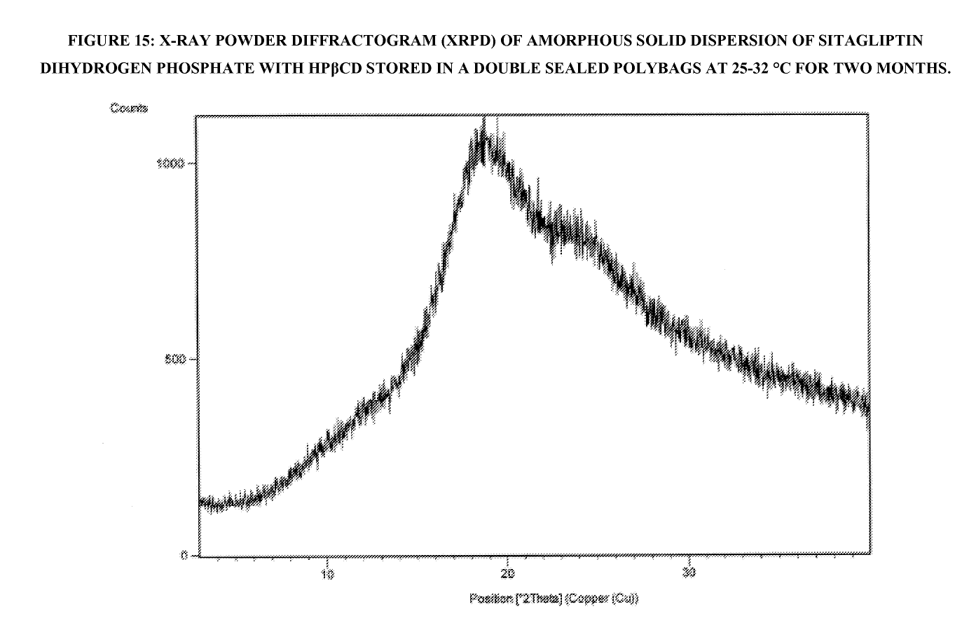

The remaining product obtained as per Example 5 was stored in a double-sealed polybags at 25° C. to 32° C. for two months to evaluate the stability. The XRPD pattern of the product stored in a double sealed polybag at 25° C. to 32° C. for two months is depicted in FIG. 15.

### Example 6

### Preparation of Amorphous Solid Dispersion of Sitagliptin Dihydrogen Phosphate with HPβCD

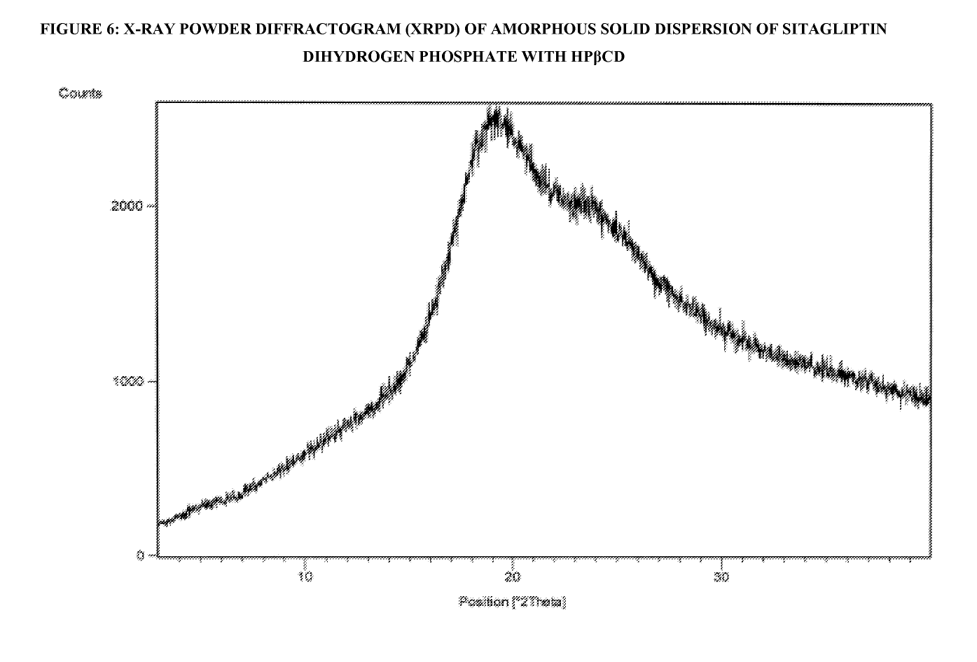

Sitagliptin dihydrogen phosphate (1.02 g) and HPβCD (0.99 g) were dissolved in methanol (60 ml). The solvent was distilled off on a Buchi rotovap set at ˜65° C. and 250 rpm under vacuum. The solid so obtained was collected and dried in a vacuum tray drier at 50° C. for 4 hours to obtain the titled compound having an XRPD pattern as depicted in FIG. 6.

### Yield: 1.61 g

### Example 7

### Preparation of Amorphous Solid Dispersion of Sitagliptin Dihydrogen Phosphate with PVP

Sitagliptin dihydrogen phosphate (5.03 g) and PVP (5.01 g) were dissolved in water (100 ml) by heating at about 65° C. The solution thus obtained was spray dried under the following conditions:

Air Inlet temperature: 130° C.

Air Outlet temperature: 54° C.

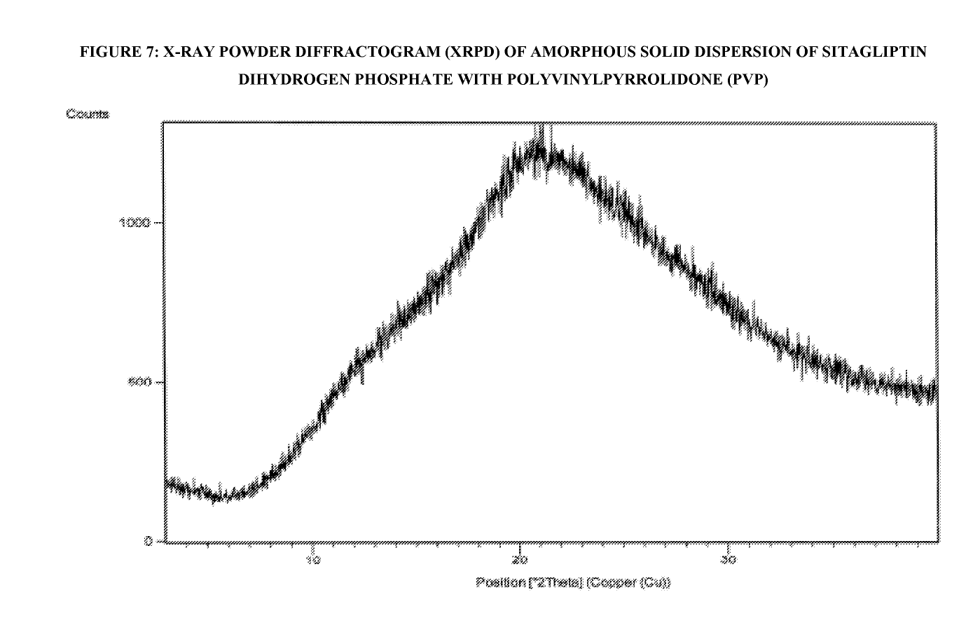

The solid so obtained was collected from the spray dryer and dried in a vacuum tray drier at 60° C. for 6 hours to obtain the titled compound having an XRPD pattern as depicted in FIG. 7.

### Yield: 5.29 g

### Example 8

### Preparation of Amorphous Solid Dispersion of Sitagliptin Dihydrogen Phosphate with PVP

Sitagliptin dihydrogen phosphate (5.15 g) and PVP (5.1 g) were dissolved in water (100 ml) by heating at about 65° C. The solution thus obtained was spray dried under the following conditions:

Air Inlet temperature: 130° C.

Air Outlet temperature: 61° C.

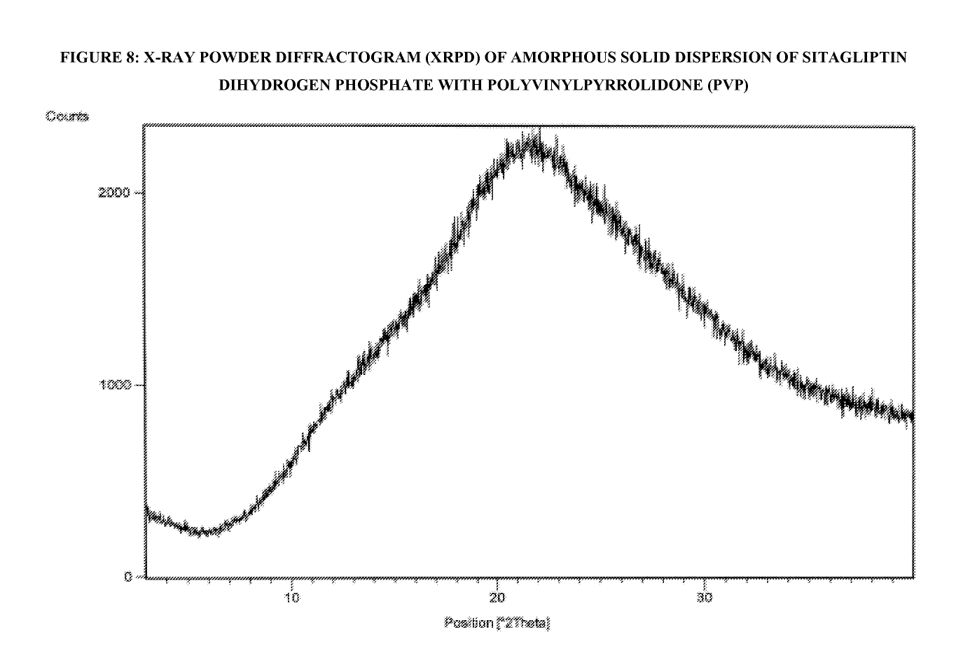

The solid so obtained was collected from the spray dryer and dried in a vacuum tray drier at 60° C. for 6 hours to obtain the titled compound having an XRPD pattern as depicted in FIG. 8.

### Yield: 5.24 g

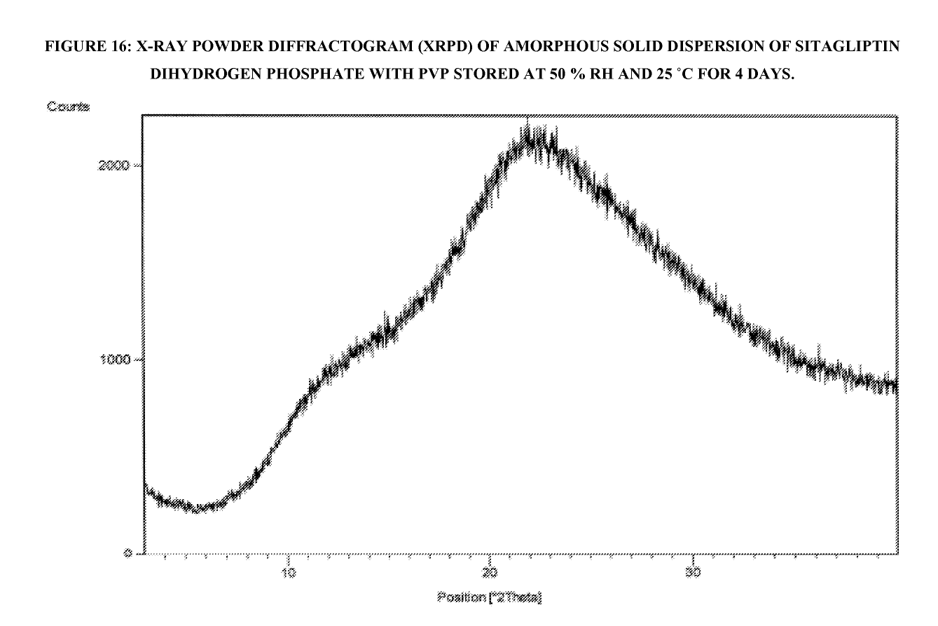

0.51 g of the product obtained as per Example 8 was stored in double-sealed polybags in a humidity chamber maintained at 50% RH and 25° C. for 4 days to evaluate the stability. The XRPD pattern of the product stored at 50% RH and 25° C. for 4 days is depicted in FIG. 16.

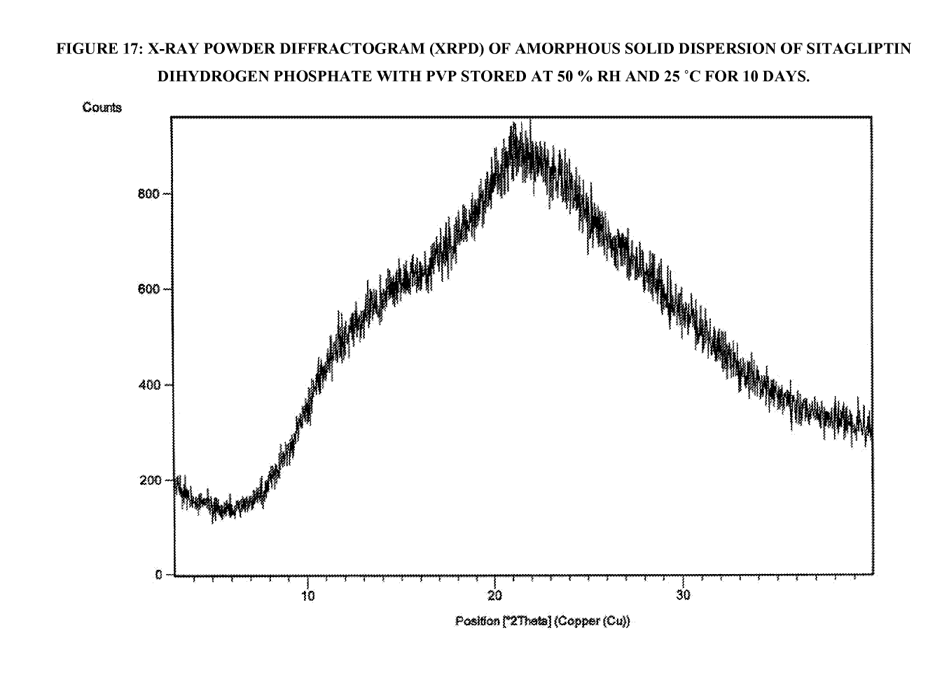

0.50 g of the product obtained as per Example 8 was stored in double-sealed polybags in a humidity chamber maintained at 50% RH and 25° C. for 10 days to evaluate the stability. The XRPD pattern of the product stored at 50% RH and 25° C. for 10 days is depicted in FIG. 17.

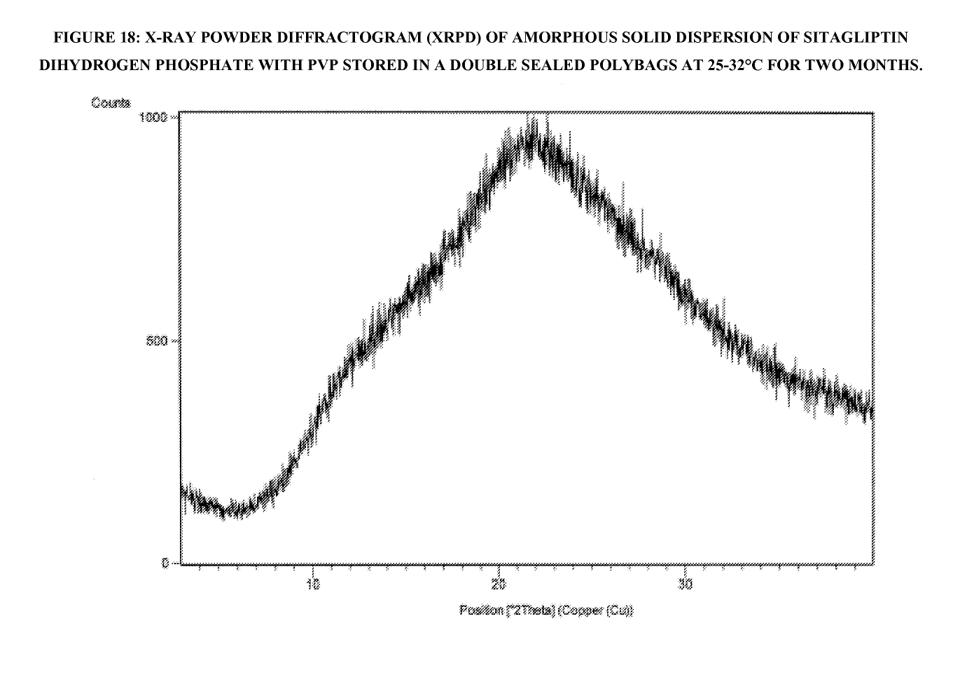

The remaining product obtained as per Example 8 was stored in double-sealed polybags at 25° C. to 32° C. for two months to evaluate the stability. The XRPD pattern of the product stored in double sealed polybags at 25° C. to 32° C. for two months is depicted in FIG. 18.

### Example 9

### Preparation of Amorphous Solid Dispersion of Sitagliptin Dihydrogen Phosphate with PVP

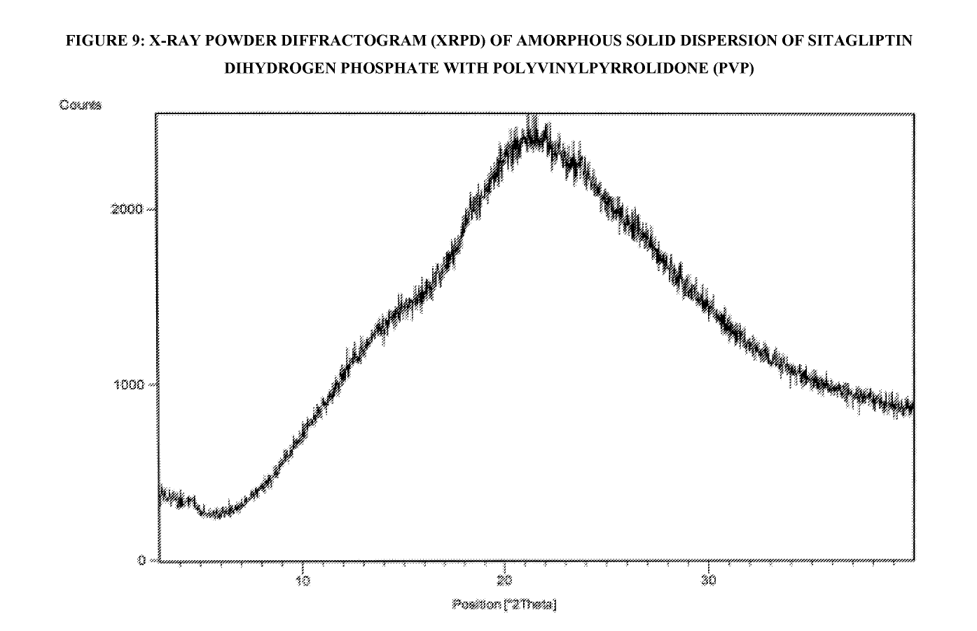

Sitagliptin dihydrogen phosphate (1.5 g) and PVP (1.01 g) were dissolved in water (40 ml). The solvent was distilled off on a Buchi rotovap set at ˜65° C. and 250 rpm under vacuum. The solid so obtained was collected and dried in a vacuum tray drier at 50° C. for 4 hours to obtain the titled compound having an XRPD pattern as depicted in FIG. 9.

Yield: 1.89 g

1. A process for the preparation of amorphous sitagliptin dihydrogen phosphate comprising the steps of:
a) obtaining a solution of sitagliptin dihydrogen phosphate;
b) removing the solvent from the solution obtained in step a) by spray drying; and
c) collecting sitagliptin dihydrogen phosphate in amorphous form.

2. The process according to claim 1, wherein the solution of sitagliptin dihydrogen phosphate is obtained by treating sitagliptin dihydrogen phosphate with one or more solvents, wherein the one or more solvents are selected from water, esters, alkanols, halogenated hydrocarbons, ketones, ethers, polar aprotic solvents or mixtures thereof.

3. (canceled)

4. (canceled)

5. (canceled)

6. (canceled)

7. (canceled)

8. (canceled)

9. (canceled)

10. The process according to claim 2, wherein the sitagliptin dihydrogen phosphate is treated with the solvent at a temperature of about 25° C. to reflux.

11. The process according to claim 2, wherein the amount of solvent is about 5 times to 20 times the quantity of sitagliptin dihydrogen phosphate.

12. The process according to claim 1, wherein step b) involves feeding the solution obtained in step a) to a spray drying apparatus having an air inlet temperature from about 70° C. to about 130° C. and an outlet temperature from about 30° C. to about 65° C.

13. A process for the preparation of amorphous sitagliptin dihydrogen phosphate comprising the steps of:
a) obtaining a solution of sitagliptin dihydrogen phosphate;
b) removing the solvent from the solution obtained in step a) by agitated thin film drying; and
c) collecting sitagliptin dihydrogen phosphate in amorphous form.

14. A process according to claim 6, wherein the solution of sitagliptin dihydrogen phosphate is obtained by treating sitagliptin dihydrogen phosphate with one or more solvents, wherein the one or more solvents are selected from the group consisting of water, esters, alkanols, halogenated hydrocarbons, ketones, ethers, polar aprotic solvents, and mixtures thereof.

15. (canceled)

16. (canceled)

17. (canceled)

18. (canceled)

19. (canceled)

20. (canceled)

21. (canceled)

22. The process according to claim 7, wherein the sitagliptin dihydrogen phosphate is treated with the solvent at a temperature of about 25° C. to reflux.

23. The process according to claim 7, wherein the amount of solvent is about 5 times to 20 times the quantity of sitagliptin dihydrogen phosphate.

24. The process according to claim 6, wherein step b) involves feeding the solution obtained in step a) to an agitated thin film dryer and removing the solvent from the solution by agitated thin film drying by heating at a temperature of about 35° C. or above.

25. (canceled)

26. A solid dispersion of sitagliptin dihydrogen phosphate.

27. The solid dispersion of claim 11 in amorphous form.

28. The solid dispersion of claim 11, comprising one or more pharmaceutically acceptable carriers.

29. The solid dispersion of claim 13, wherein the pharmaceutically acceptable carrier is polyvinyl pyrrolidone (PVP) or hydroxypropyl-β-cyclodextrin (HPβCD).

30. The solid dispersion of claim 13, wherein the amount of sitagliptin dihydrogen phosphate is from about 0.1% to about 95% by relative weight to the total weight of the solid dispersion.

31. A solid dispersion of sitagliptin dihydrogen phosphate with HPβCD in amorphous form.

32. The amorphous solid dispersion of claim 16, having a characteristic XRD pattern substantially as depicted in FIG. 4, FIG. 5 or FIG. 6.

33. (canceled)

34. (canceled)

35. The amorphous solid dispersion of claim 16, which is stable for at least 4 days when exposed to a temperature of about 25° C. and a relative humidity of about 50% and has a characteristic XRD pattern substantially as depicted in FIG. 13.

36. The amorphous solid dispersion of claim 16, which is stable for at least 10 days when exposed to temperature of about 25° C. and a relative humidity of 50% and has a characteristic XRD pattern substantially as depicted in FIG. 14.

37. The amorphous solid dispersion of claim 16, which is stable for at least two months when kept in double sealed polybags at about 25° C. to 32° C. and has a characteristic XRD pattern substantially as depicted in FIG. 15.

38. A solid dispersion of sitagliptin dihydrogen phosphate with polyvinylpyrrolidone (PVP) in amorphous form.

39. The solid dispersion of claim 21, having a characteristic XRD pattern substantially as depicted in FIG. 7, FIG. 8 or FIG. 9.

40. (canceled)

41. (canceled)

42. The solid dispersion of claim 21, which is stable for at least 4 days when exposed to a temperature of about 25° C. and a relative humidity of about 50% and has a characteristic XRD pattern substantially as depicted in FIG. 16.

43. The solid dispersion of claim 21, which is stable for at least 10 days when exposed to a temperature of about 25° C. and a relative humidity of 50% and has a characteristic XRD pattern substantially as depicted in FIG. 17.

44. The solid dispersion of claim 21, which is stable for at least two months when kept in double sealed polybags at about 25° C. to 32° C. and has a characteristic XRD pattern substantially as depicted in FIG. 18.

45. A process for the preparation of a solid dispersion of sitagliptin dihydrogen phosphate comprising:
a) combining sitagliptin dihydrogen phosphate with one or more pharmaceutically acceptable carriers; and
b) isolating the solid dispersion of amorphous sitagliptin dihydrogen phosphate.

46. The process according to claim 26, wherein combining the sitagliptin dihydrogen phosphate with one or more pharmaceutically acceptable carriers includes at least one of the steps of adding, dissolving, slurrying, or stirring in a solvent at a temperature of about 25° C. to reflux.

47. The process according to claim 27, wherein the solvent is selected from the group consisting of water, esters, alkanols, halogenated hydrocarbons, ketones, ethers, polar aprotic solvents, or mixtures thereof.

48. (canceled)

49. (canceled)

50. (canceled)

51. (canceled)

52. (canceled)

53. (canceled)

54. The process according to claim 26, wherein the pharmaceutically acceptable carrier is polyvinyl pyrrolidone (PVP), or hydroxypropyl-β-cyclodextrin (HPβCD).

55. The process according to claim 26, wherein step b) involves spray drying, lyophilization, agitated thin film drying or melt extrusion.

56. A method of treating or preventing Type 2 diabetes mellitus comprising administering to a patient in need thereof a therapeutically effective amount of solid dispersion of sitagliptin dihydrogen phosphate.
